(19) United States
(12) Patent Application Publication
(10) Pub. No.: US 2002/0029430 A1
(43) Pub. Date: Mar. 14, 2002

(54) Device for hair coloring, styling & permanent waving

(76) Inventor: Kim DeSoto, Towson, MD (US)

Correspondence Address: Max Stul Oppenheimer, P.O.Box 50, Stevenson, MD, 21153, US

(21) Appl. No.: 09/949,345

(22) Filed: Sep. 7, 2001

Related U.S. Application Data
(60) Provisional application No. 60/231,455, filed on Sep. 8, 2000.

(51) Int. Cl. A61K 7/13

(52) U.S. Cl. 8/405

(57) ABSTRACT

A lightweight closed-cell plastic (such as polyethylene) foam roller can be used in the hair color, hair permanent waving and hair styling industry, allowing the hairdresser many choices for the customizing of hair color, specifically for hair with curl. The roller allows hair treatment agents (such as coloring, waving and styling agents) to be isolated and stabilized and allows full development time of the treatment agents and also allows the hair colorist to apply two or more color agents on the strands of hair to achieve a three dimensional effect.

This application is entitled to, and claims the benefit of, priority from U.S. Provisional Application Ser. No. 60/231,455, filed Sep. 8, 2000.

### FIELD AND BACKGROUND OF THE INVENTION

1. Field of the Invention

The present invention relates in general to the beauty industry and in particular to devices for hair treatment, including coloring, permanent waving and hair styling.

2. Background Information

The invention described and claimed herein comprises a novel device for hair treatment, of particular application to treatment of curly or textured human hair.

Curly or textured hair poses special problems for the application of two or more hair coloring agents (as would be required, for example, in creating a highlight effect), particularly how to stabilize the products on the hair long enough to allow proper processing times, not have all the hair strands collapse on themselves or have the coloring agents run together.

In order for the coloring agents to work properly, they must be applied to strands of hair that have been wrapped around a device. Coloring agents are then saturated onto the strands in the proper areas on the device. The coloring agents are then covered with a plastic wrap so as not to allow for the migration of the coloring agents or the displacement (having the coloring agents rub off onto surrounding hair or devices) of color.

Typical devices, such as wooden dowel rods and standard hard plastic rollers do not produce satisfactory results.

When using these devices, once the styling, coloring or permanent waving product has been applied, the hair is covered with an aluminum foil to hold the product in place. Dowel rods and aluminum foil do not ensure that the product will not run, blend into, infiltrate or completely mix together once the foil is folded together. Because the aluminum foil is opaque, it is not possible to determine if coloring agents have been placed and pressed together properly, nor is it possible to check the development of the color with the aluminum foil covering the product.

Standard styling rollers are not designed for proper attachment to the base of the hair at the scalp that would allow for the application of hair coloring agents. The styling rollers are too narrow to accommodate most lengths of hair. Attempts to use styling rollers did not produce satisfactory results; they were too short in length and could not be fastened to the scalp properly to insure the proper placement of the coloring agents.

Permanent waving rods are not designed for use with coloring agents nor are they effective in securing the hair at the scalp when applying coloring agents. The rods are also too heavy and are not long enough for proper color application technique. In experiments using such rods, the rods fell out of the hair, pulled the coloring agents off the hair, or the plastic band which secures the rod pulled the agents off the strands.

Hair swags or rats are sometimes made from cloth, in which case the coloring agents are absorbed into the cloth and proper processing of the coloring agents can not occur. If the swag or rat is made from synthetic materials it is not in the proper form or size to create the necessary highlight effects. It also lacks the control necessary for the surface area to remain undisturbed in order for the coloring agents to develop.

### SUMMARY OF THE INVENTION

The foregoing problems are overcome, and other advantages are provided by a hair processing roller comprised of a closed cell plastic foam, preferably polyethylene, with at least one, and preferably two or more thin strips of hook fasteners (such as the open, or hook end of VELCRO (TM) hook and loop fasteners, attached on opposing sides of the roller. Optionally, fastening means are provided at one end of the roller, for securing strands of hair to be treated.

It is an object of the invention to provide a hair processing roller which provides improved coloring of curly or textured human hair.

It is another object of the invention to provide a hair processing roller which will allow two or more coloring agents to be applied to hair and especially curly/textured hair that allows a stable inert surface that is secure to the head.

It is another object of the invention to provide a reusable roller that is chemically inert with respect to hair processing products, is easy to clean and is reusable.

It is another object of the invention to provide a hair processing roller of a material that can be cut easily, so that it can be customized for the length of hair it is being used on.

It is another object of the invention to provide a hair processing roller which is sufficiently lightweight to avoid collapsing the hair which is being treated.

It is another object of the invention to provide a hair processing roller which gives the hair colorist the ability to isolate, stabilize and separate two or more coloring agents on strands of hair.

It is another object of the invention to provide a guideline or reference point for placement of the hair processing compounds.

A principal feature of the invention is the use of a closed cell plastic material.

Another principal feature of the invention is the use of hook fasteners for securing the hair to the roller in sufficient contact to permit stable development.

Among the advantages of the invention are the improved results when highlighting curly or textured hair.

The various features of novelty which characterize the invention are pointed out with particularity in the claims annexed to and forming a part of this disclosure. For a better understanding of the invention, its advantages and objects, reference is made to the accompanying drawings and descriptive matter in which a preferred embodiment of the invention is illustrated.

### BRIEF DESCRIPTION OF THE DRAWINGS

The foregoing and still other objects of this invention will become apparent, along with various advantages and features of novelty residing in the present embodiments, from study of the following drawing, in which:

FIG. 1 is a perspective view of the device.

FIG. 2 is a perspective view of the device, rotated 90° from the view in FIG. 1.

FIG. 3 is an end-on view of the device, viewed from its distal end.

FIG. 4 is a view of an alternative embodiment.

### DESCRIPTION OF THE PREFERRED EMBODIMENT

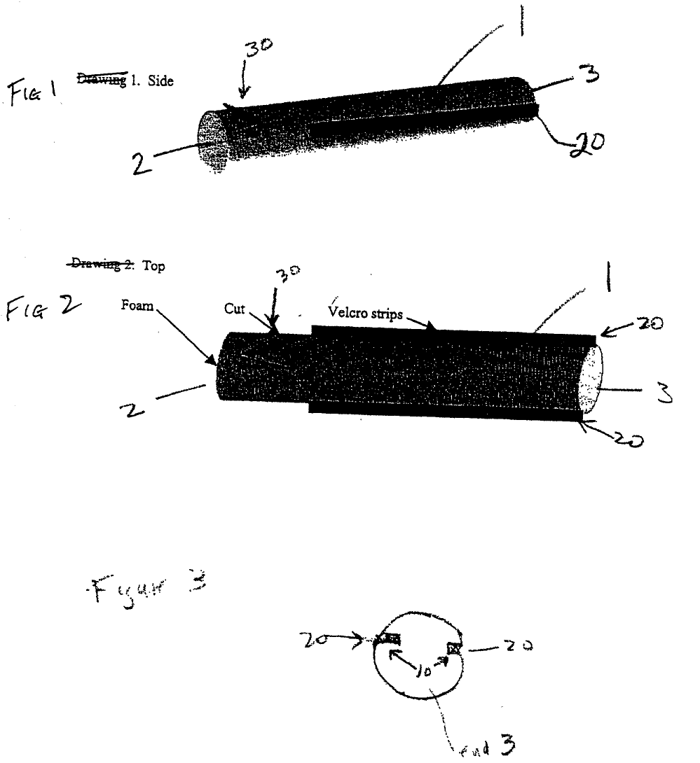

Referring to the drawings, the invention is a novel device for treatment of human hair, shown in overview in FIG. 1, characterized by a lightweight, inert tube (1) of closed cell material, having a proximal end (2) and a distal end (3). Near the proximal end (2), securing means for securing strands of hair are provided; extending from at or near the distal end (3) a longitudinal strip (20) of lightweight means for securing strands of hair, preferably a hook material (such as the hook end of VELCRO TM hook and loop material), is provided.

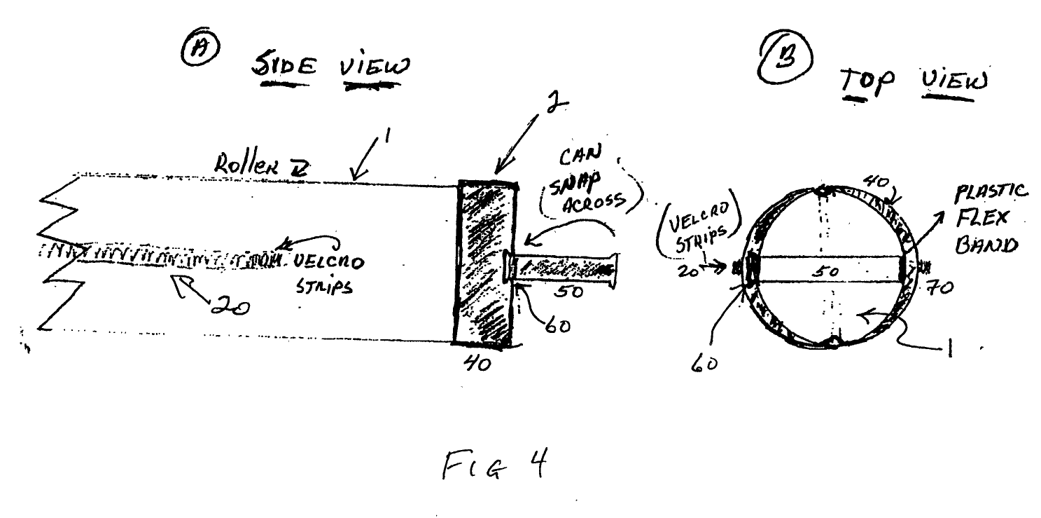

A prototype device according to the invention may be made as follows. A tube (1) of suitable diameter (¾ inch in the prototype), made from a flexible, closed cell plastic (preferably polyethylene) is selected and cut to a length approximately equal to the length of the hair to be treated. Two small longitudinal grooves (10), of a width and depth which accommodates the strip or strips of hook material so that the hooks extend slightly above the surface of the tube (1), preferably approximately 1/16 inch wide and 1/32 inch deep, are made at opposite sides of the tube (1) as shown in FIG. 3, starting at the distal end (3) and running longitudinally for the most of the length of the tube (1). Strips (20) of a hook and loop material such as VELCRO (TM) are inserted into the grooves (10) and attached (preferably with a thermal adhesive), although in an alternative embodiment the strips (20) could be surface mounted to the tube (1). Next, securing means (30) are created. In the prototype, a V-shaped (when viewed from above, as in FIG. 2) cut was made near the proximal end (2) of the tube (1), so as to create a hinged flap into which a strand of hair could be placed, although alternatively hook and loop material could be fastened in that position.

An alternative securing means is illustrated in FIG. 4. Again located at the proximal end (2) of the tube (1), the alternative securing means comprises a ring (40) tightly fitted to or fastened to the tube (1), to which a flexible band (preferably plastic) (50) is attached by a hinge (20) at one end, and releasably attached by attachment means (preferably a snap fastener) (70). In operation, the attachment means (70) is released and the band (50) is swung open, a strand of hair is placed across ring (40), the band (50) is swung closed, thereby securing the strand of hair in place, and the band is fastened in place by attachment means (70).

The range of uses of the invention are illustrated in the following examples.

Color Application:

First, select a group of hair strands that on the scalp comprises by ½ inch. The strands should be about 6 inches or longer. Place the base of the strands into securing means (30) of the tube (1) (i.e., near the scalp). The remainder of the strands are then wound around the tube (1), securing the ends of the strands by strips (20). Coloring agents are then applied to the tube (1) and hair. Optionally, the base of the tube (1) at the securing means (30) can have a clip (50) (a suitable clip would be a clothespin) applied to grasp or secure the tube (1) and hair to the hair at the base of the scalp. Finally, plastic wrap is placed around both the tube and coloring agents. The invention when used in this manner allows the colorist to apply two or more colors to one strand of hair. This creates a multitude of options in color design on the hair strands. This would allow the colorist to recreate a very natural look in curly hair or a very avant-garde creation. For example, it is possible to apply color to select areas of hair so as to create a three-dimensional effect.

Color Application—Reference Point.

Typical rollers for hair coloring are cylindrical, with no defining features. The present invention has the defining feature provided by the strip or strips, which may be used to define a “top” and “bottom” of the roller. This allows the haircolorist a “guide” or reference points in which to place the coloring agents.

Styling or Finishing Application:

The invention may also be used for styling purposes, allowing a stylist to obtain a spiral curl affect to the hair. First the Stylist selects a small amount of hair ¼ of an inch by ½ inch, then opens the base end of the roller, attaches the roller to the base of the strands and wraps the hair around the roller. Any setting gels or lotions may be used to adhere the strands of hair to the tube. The hair is then dried, styled and hair spray applied if necessary. For this particular use in styling, the Velcro strips are not used. The finished style is a spiral-curled affect.

Permanent Waving Application:

The invention can be applied to the base of the hair strand as described above under “color application”. The hair is then wound around tube (1) and fastened by the hook and loop strips (20). The hair is then saturated with a permanent wave solution and the perming process continues from that point as normal, according to the permanent solution manufacturer's directions, thereby creating a spiral-curled effect.

Thus, there has been described a novel device for treatment of hair, that has a number of novel features, and a manner of making and using the invention.

While a specific embodiment of the invention has been shown and described in detail to illustrate the application of the principles of the invention, it will be understood that the invention may be embodied otherwise without departing from such principles and that various modifications, alternate constructions, and equivalents will occur to those skilled in the art given the benefit of this disclosure. Thus, the invention is not limited to the specific embodiment described herein, but is defined by the appended claims.

What is claimed is:

1. A device for treatment of human hair, comprising a closed cell plastic foam roller having two ends defining a longitudinal tube, said device further comprising a thin strip of hook fastener material running longitudinally along said tube.

2. A device as in claim 1 further comprising attachment means for securing a strand of human hair.

3. A device as in claim 2 wherein said attachment means comprise cutouts in one of said ends, suitable for receiving strands of human hair.

4. A device as in claim 2 wherein said attachment means comprise a ring secured at one of said ends, and a flexible band having a first end and a second end, said flexible band being attached to said ring at its first end a hinge and being releasably attached to said ring at its second end by release means.
